(19) United States
(12) Patent Application Publication
(10) Pub. No.: US 2024/0076631 A2
(43) Pub. Date: Mar. 7, 2024

(54) METHOD FOR REMOVING HOST CELL DNA FROM VIRUS PREPARATION

(71) Applicant: Takeda Vaccines, Inc., Cambridge, MA (US)

(72) Inventors: Yock LEE, Singapore (SG); Jia LOH, Singapore (SG); Joseph SANTANGELO, Newton, MA (US)

(73) Assignee: Takeda Vaccines, Inc., Cambridge, MA (US)

(21) Appl. No.: 17/802,443

(22) PCT Filed: Feb. 26, 2021
(86) PCT No.: PCT/US2021/019991

§ 371 Date: Aug. 25, 2022

(30) Foreign Application Priority Data
Feb. 27, 2020 (EP) 20159895.0

(51) Int. Cl. C12N 7/00; B01D 15/36

(52) U.S. Cl. CPC C12N 7/00; B01D 15/363; C12N 2770/24123; C12N 2770/24151

(57) ABSTRACT

The present invention relates to a method for removing host cell DNA from a sample comprising infectious viral particles and host cell DNA by anion exchange chromatography in the presence of at least one of a non-ionic surfactant, a sugar and a protein and to a method for purifying recombinant infectious viral particles from a host cell culture employing such an anion exchange chromatography step.

### FIELD OF THE INVENTION

The present invention relates to a method for removing host cell DNA from a sample comprising infectious viral particles and host cell DNA by anion exchange chromatography in the presence of at least one of a non-ionic surfactant, a sugar and a protein and to a method for purifying recombinant infectious viral particles from a host cell culture employing such an anion exchange chromatography step.

### BACKGROUND OF THE INVENTION

Vaccines for protection against viral infections have been effectively used to reduce the incidence of human disease. One of the most successful technologies for viral vaccines is to immunize animals or humans with a weakened or attenuated virus strain (a “live attenuated virus”). Due to limited replication after immunization, the attenuated virus strain does not cause disease. However, the limited viral replication is sufficient to express the full repertoire of viral antigens and can generate potent and long-lasting immune responses to the virus. Thus, upon subsequent exposure to a pathogenic virus strain, the immunized individual is protected from the disease. These live attenuated viral vaccines are among the most successful vaccines used in public health.

For producing live attenuated viruses to be included into a vaccine, typically mammalian host cells such as Vero cells are infected with the virus which then replicates within the cells, leading to the secretion of infectious viral particles into the cell supernatant. These infectious viral particles then have to be separated from impurities such as host cell proteins and host cell DNA, before they can be used to immunize subjects.

WO 02/024876 A2 discloses a process for the manufacture of a whole-virus vaccine wherein infected host cells are incubated in a serum-free culture medium in the presence of a nuclease and a protease.

WO 2006/22964 A1 describes the purification of an inactivated West Nile Virus by affinity chromatography using Cellufine sulfate chromatography.

WO 2013/106337 A1 discloses a method for purifying recombinant infectious flavivirus viral particles by tangential flow filtration followed by anion exchange chromatography performed in bind-and-elute mode.

WO 2015/092287 A1 describes a method for purifying enveloped viruses by performing an anion exchange chromatography step using an acidic buffer having a pH of less than 6.0. It also describes that the application of a tangential flow filtration step before anion exchange chromatography results in a substantial improvement of the purification efficiency of the enveloped virus.

Nevertheless, there is still a need for methods which allow the efficient removal of host cell DNA and proteins from the viral particles to be used in a vaccine.

### SUMMARY OF THE INVENTION

The present invention provides a process for the efficient removal of host cell DNA from a sample comprising infectious viral particles and host cell DNA which minimizes the loss of virus titer during the removal of host cell DNA.

Accordingly, the present invention relates to a method for removing host cell DNA from a sample comprising infectious viral particles and host cell DNA, comprising the steps of:
(a) mixing said sample comprising infectious viral particles and host cell DNA with a composition comprising one or more of a non-ionic surfactant, a sugar and a protein, thereby providing a mixture; and
(b) subjecting the mixture of step (a) to anion exchange chromatography.

In one embodiment, the non-ionic surfactant is a nonionic triblock copolymer such as an ethylene oxide propylene oxide (EO-PO) block copolymer. In one embodiment, the non-ionic surfactant is poloxamer 407 or poloxamer 403. In one embodiment, the concentration of the non-ionic surfactant in the mixture of step (a) is 0.05% to 0.5% (w/v).

In one embodiment, the sugar is selected from trehalose, glucose, sucrose, fructose and maltose, preferably is trehalose. In one embodiment, the concentration of the sugar in the mixture of step (a) is 1% to 7.5% (w/v).

In one embodiment, the protein is an albumin, preferably is human serum albumin. In one embodiment, the concentration of the protein in the mixture of step (a) is 0.005% to 0.05% (w/v).

In one embodiment, the composition comprises poloxamer 407, trehalose dihydrate and human serum albumin. In one embodiment, the mixture of step (a) comprises 0.2% (w/v) poloxamer 407, 3% trehalose dihydrate and 0.02% human serum albumin.

In one embodiment, the anion exchange chromatography is operated in flow-through mode. In one embodiment, the anion exchange chromatography uses an anion exchange chromatography membrane. In one embodiment, the anion exchange chromatography membrane comprises quaternary ammonium groups.

In one embodiment, the infectious viral particles are enveloped infectious viral particles. In one embodiment, the infectious viral particles are infectious flavivirus viral particles. In one embodiment, the infectious viral particles are infectious dengue virus viral particles. In one embodiment, the infectious dengue virus viral particles are infectious live attenuated dengue virus viral particles or infectious chimeric dengue virus viral particles.

In one embodiment, less than 5% of the viral titer are lost during the anion exchange chromatography.

In one embodiment, the anion exchange chromatography results in an at least tenfold reduction of the host cell DNA content.

In one embodiment, the method does not include a step of treating the sample comprising infectious viral particles and host cell DNA with an endonuclease.

The present invention also relates to a method for purifying infectious viral particles from a host cell culture, comprising the steps of:
(a) harvesting the host cell culture supernatant containing infectious viral particles from said host cell culture;
(b) mixing the harvested infectious viral particles with a composition comprising one or more of a non-ionic surfactant, a sugar and a protein, thereby providing a mixture;
(c) subjecting the mixture of step (b) to one or more anion exchange chromatography steps and collecting the flow-through;
(d) subjecting the flow-through from step (c) to tangential flow filtration and collecting the retentate; and
(e) recovering the purified infectious viral particles from the retentate of step (d).

In one embodiment, the non-ionic surfactant is a nonionic triblock copolymer, such as an ethylene oxide propylene oxide (EO-PO) block copolymer. In one embodiment, the non-ionic surfactant is poloxamer 407 or poloxamer 403. In one embodiment, the concentration of the non-ionic surfactant in the mixture of step (a) is 0.05% to 0.5% (w/v).

In one embodiment, the sugar is selected from trehalose, glucose, sucrose, fructose and maltose, preferably is trehalose. In one embodiment, the concentration of the sugar in the mixture of step (a) is 1% to 7.5% (w/v).

In one embodiment, the protein is an albumin, preferably is human serum albumin. In one embodiment, the concentration of the protein in the mixture of step (a) is 0.005% to 0.05% (w/v).

In one embodiment, the composition comprises poloxamer 407, trehalose and human serum albumin. In one embodiment, the composition comprises 0.2% (w/v) poloxamer 407, 3% trehalose dihydrate and 0.02% human serum albumin.

In one embodiment, the anion exchange chromatography uses an anion exchange chromatography membrane. In one embodiment, the anion exchange chromatography membrane comprises quaternary ammonium groups.

In one embodiment, the infectious viral particles are enveloped infectious viral particles. In one embodiment, the infectious viral particles are infectious flavivirus viral particles. In one embodiment, the infectious viral particles are infectious dengue virus viral particles. In one embodiment, the infectious dengue virus viral particles are infectious live attenuated dengue virus viral particles or infectious chimeric dengue virus viral particles.

In one embodiment, the method does not include a step of treating the sample comprising infectious viral particles and host cell DNA with an endonuclease.

In one embodiment, the host cell culture is a Vero cell culture.

In one embodiment, after harvesting the host cell culture supernatant comprising the infectious viral particles is subjected to depth filtration. In one embodiment, the depth filtration is through a 0.45 μm and a 0.2 μm filter.

In one embodiment, the tangential flow filtration comprises filtration through one or more TFF membrane cassettes. In one embodiment, the one or more TFF membrane cassettes comprise a cellulose media.

### DETAILED DESCRIPTION OF THE INVENTION

Where the term “comprise” or “comprising” is used in the present description and claims, it does not exclude other elements or steps. For the purpose of the present invention, the term “consisting of” is considered to be an optional embodiment of the term “comprising”. If hereinafter a group is defined to comprise at least a certain number of embodiments, this is also to be understood to disclose a group which optionally consists only of these embodiments.

Where an indefinite or a definite article is used when referring to a singular noun e.g. “a” or “an”, “the”, this includes a plural form of that noun unless specifically stated. Vice versa, when the plural form of a noun is used it refers also to the singular form.

Furthermore, the terms first, second, third or (a), (b), (c) and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In the context of the present invention any numerical value indicated is typically associated with an interval of accuracy that the person skilled in the art will understand to still ensure the technical effect of the feature in question. As used herein, the deviation from the indicated numerical value is in the range of ±10%, and preferably of ±5%. The aforementioned deviation from the indicated numerical interval of ±10%, and preferably of ±5% is also indicated by the terms “about” and “approximately” used herein with respect to a numerical value.

By the method of the present invention host cell DNA is removed from a sample comprising infectious viral particles and host cell DNA. The term “removing host cell DNA” means that the content of host cell DNA after the method of the invention has been performed is lower than the content of host cell DNA before the method of the invention is performed. In one embodiment, the method of the present invention results in an at least tenfold reduction of the host cell DNA content. Preferably, the method of the present invention results in a reduction of the host cell DNA content by at least 12-fold or 15-fold, more preferably by at least 18-fold or 20-fold and most preferably by at least 22-fold. The fold reduction can be calculated by dividing the host cell DNA content in the sample before the method is performed with the host cell DNA content in the sample after the method has been performed.

Preferably, the content of host cell DNA after the method of the invention has been performed is lower than the detection limit of the assay used for determining the host cell DNA content. In one embodiment, the content of host cell DNA after the method of the invention has been performed is 50 ng/ml or lower.

Methods to determine the host cell DNA content in a biological sample obtained from a host cell are known in the art and include quantitative PCR using primers which specifically bind to the host cell DNA, but not to the viral nucleic acids. Kits for determining host cell DNA content are commercially available for example from ThermoFisher. Preferably, the Picogreen® dye is used to determine the host cell DNA content.

The “host cell DNA” is derived from the cell in which the infectious viral particles were produced. The host cell DNA can be distinguished from the viral nucleic acids by its nucleic acid sequences which differ from those of the viral nucleic acids. In one embodiment, Vero cells are used to produce infectious viral particles. Accordingly, in this embodiment the host cell DNA is derived from the Vero cells and is also called “Vero cell DNA”. The Vero cell DNA can be distinguished from the viral nucleic acids by its nucleic acid sequences which differ from those of the viral nucleic acids.

The term “infectious viral particles” means that the virus is able to infect suitable host cells such as Vero cells and replicate in these host cells, leading to the production of additional infectious viral particles. The virus does not lose the ability to infect suitable host cells when the method of the present invention is performed so that the purified viral particles obtained by the method of the present invention are still able to infect suitable host cells. Accordingly, the viral particles obtained by the method of the present invention are infectious viral particles. In particular, the method of the present invention does not comprise a step of inactivating the viral particles. A step of inactivating the viral particles, e.g. by treatment with formalin or beta-propiolactone, leads to the loss of infectivity of the viral particles. The term “infectious viral particles” does not comprise viral-like particles which are not infectious. In one embodiment, the term “infectious viral particles” comprises live attenuated viruses.

In one embodiment, the infectious viral particles are derived from enveloped viruses. Examples of enveloped viruses include, but are not limited to, poxviruses, orthomyxoviruses, paramyxoviruses, and flaviviruses.

An enveloped virus refers to a viral particle that has an outer wrapping or envelope derived from the infected host cell in a budding process whereby the newly formed virus particles become wrapped in an outer coat that is made from a small piece of the cell's plasma membrane. The budding process by which the virus acquires its envelope results in the viral particles being expelled from the host cells used to grow the virus.

Examples of enveloped orthomyxoviruses include, but are not limited to, the influenza type A viruses including but not limited to the strains H1N1, H1N2, H2N2, H3N1, H3N2, H3N8, H5N1, H5N2, H5N3, H5N8, H5N9, H7N1, H7N2, H7N3, H7N4, H7N7, H9N2, and H10N7.

Examples of enveloped paramyxoviruses include, but are not limited to, human respiratory syncytial virus, measles virus, and mumps virus.

In one particular embodiment, the enveloped viruses include viruses of the genus flavivirus. The genus flavivirus comprises enveloped positive-strand RNA viruses such as West Nile (WN) virus, Japanese Encephalitis virus (JEV), Zika virus, Dengue fever virus, yellow fever virus (YF), Kyasanur Forest disease virus, Murray Valley encephalitis virus, St. Louis encephalitis virus, Tick-borne encephalitis virus, West Nile encephalitis virus, Central European encephalitis (TBE-W) virus, Far Eastern encephalitis (TBE-FE) virus, Kunjin virus, Tyuleniy virus, Ntaya virus, Uganda S virus, Modoc virus, BVDV (e.g, strains NADL, and 890), CSFV Alfort 187, BDV BD31 virus, and/or GB virus-A, -B, and/or -C.

In one particular embodiment, the infectious viral particle is an infectious dengue virus viral particle. The dengue virus is a single stranded, positive sense RNA virus of the family flaviviridae. The taxonomy is outlined in Table 1. The family flaviviridae includes three genera, flavivirus, hepacivirus and pestivirus. The genus flavivirus contains highly pathogenic and potentially hemorrhagic fever viruses, such as yellow fever virus and dengue virus, Zika virus, encephalitic viruses, such as Japanese encephalitis virus, Murray Valley encephalitis virus and West Nile virus, and a number of less pathogenic viruses.

TABLE 1
Dengue Virus Taxonomy of the GMO Parental Strain

| | |
|---|---|
| Family | Flaviviridae |
| Genus | Flavivirus |
| Species | Dengue virus |
| Strains | Dengue Serotype 2 (Strain 16681), Strain DEN-2 PDK-53 |
| GMO parent | TDV-2 |

The flavivirus genome comprises in 5′ to 3′ direction:
a 5′-noncoding region (5′-NCR),
a capsid protein (C) encoding region,
a pre-membrane protein (prM) encoding region,
an envelope protein (E) encoding region,
a region encoding nonstructural proteins (NS1, NS2A, NS2B, NS3, NS4A, NS4B, NS5) and
a 3′ noncoding region (3′-NCR).

The viral structural proteins are C, prM and E, and the nonstructural proteins are NSI to NS5. The structural and nonstructural proteins are translated as a single polyprotein and processed by cellular and viral proteases.

As used herein, the term “dengue serotype” refers to a species of dengue virus which is defined by its cell surface antigens and therefore can be distinguished from other dengue serotypes by serological methods known in the art. At present, four serotypes of dengue virus are known, i.e. dengue serotype 1 (DENY-1), dengue serotype 2 (DENV-2), dengue serotype 3 (DENV-3) and dengue serotype 4 (DENV-4).

In one particular embodiment, the infectious viral particle is a live attenuated dengue virus. As used herein, the term “live attenuated dengue virus” refers to a viable and infectious dengue virus which is mutated to provide reduced virulence. The live attenuated dengue virus can be a dengue virus in which all components are derived from the same dengue serotype or it can be a chimeric dengue virus having parts from two or more dengue serotypes.

In one embodiment, the live attenuated dengue virus is a molecularly characterized and cloned dengue serotype 2 strain derived from the live attenuated DEN-2 PDK-53 virus strain (TDV-2). This attenuated TDV-2 strain was generated by cDNA cloning of the attenuated laboratory-derived DEN-2 PDK-53 virus strain that was originally isolated at Mahidol University, Bangkok, Thailand (Kinney et al. (1997) Virology 230(2): 300-308). DEN-2 PDK-53 was generated by 53 serial passages in primary dog kidney (PDK) cells at 32° C. (Bhamarapravati et al. (1987) Bull. World Health Organ. 65(2): 189-195). In one embodiment, TDV-2 has the nucleotide sequence according to SEQ ID No. 3 and/or the amino acid sequence according to SEQ ID No. 4.

The attenuated DEN-2 PDK-53 strain (the precursor of TDV-2) was derived from the wild type virus strain DEN-2 16681 and differs in nine nucleotides from the wild type as follows (Kinney et al. (1997) Virology 230(2): 300-308):
(i) 5-noncoding region (NCR)-57 (nt-57 C-to-T): major attenuation locus
(ii) prM-29 Asp-to-Val (nt-524 A-to-T)
(iii) nt-2055 C-to-T (E gene) silent mutation
(iv) NS1-53 Gly-to-Asp (nt-2579 G-to-A): major attenuation locus
(v) NS2A-181 Leu-to-Phe (nt-4018 C-to-T)
(vi) NS3-250 Glu-to-Val (nt-5270 A-to-T): major attenuation locus
(vii) nt-5547 (NS3 gene) T-to-C silent mutation
(viii) NS4A-75 Gly-to-Ala (nt-6599 G-to-C)
(ix) nt-8571 C-to-T (NS5 gene) silent mutation

The three nucleotide changes located in the 5′ noncoding region (NCR) (nucleotide 57) (mutation (i)), the NS1 (amino acid 828 of SEQ ID NO. 4) (mutation (iv)) and NS3 genes (amino acid 1725 of SEQ ID NO. 4) (mutation (vi)) form the basis for the attenuation phenotype of the DEN-2 PDK-53 strain (Kinney et al. (1997) Virology 230: 300-308; Butrapet et al. (2000) J. Virol. 74(7): 3111-3119).

A “virus strain” and in particular a “dengue virus strain” is a genetic subtype of a virus, in particular of a dengue virus, which is characterized by a specific nucleic acid sequence. A dengue serotype may comprise different strains with different nucleic acid sequences which have the same cell surface antigens and are therefore recognized by the same antibodies. A dengue virus strain can be a dengue virus in which all components are derived from the same dengue serotype or it can be a chimeric dengue virus having parts from two or more dengue serotypes.

A “chimeric virus” or “chimeric strain” or “chimeric virus strain” comprises parts from at least two different viruses. In one embodiment, the chimeric virus comprises the prM and E proteins of dengue virus and the other proteins from another flavivirus. In one embodiment, the chimeric virus comprises the prM and E proteins of dengue virus and the other proteins from another flavivirus such as yellow fever virus, Zika virus, West Nile virus, Japanese encephalitis virus, St. Louis encephalitis virus and tick-borne encephalitis virus. In one embodiment, the chimeric virus comprises the prM and E proteins of dengue virus and the other proteins from yellow fever virus strain YF-17D. Such chimeric viruses are present in the commercial product Dengvaxia® and are described in, e.g., WO 98/37911, WO 03/101397, WO 2007/021672, WO 2008/007021, WO 2008/047023 and WO 2008/065315.

A “chimeric dengue virus” or “chimeric dengue serotype strain” or “chimeric dengue strain” comprises parts from at least two different dengue serotypes. As used herein, the chimeric dengue virus does not include parts from a different flavivirus such as yellow fever virus, Zika virus, West Nile virus, Japanese encephalitis virus, St. Louis encephalitis virus and tick-borne encephalitis virus. In particular, the chimeric dengue virus described herein does not include parts from the yellow fever virus. Such chimeric dengue viruses are described in WO 01/060847 A2, WO 2014/150939 A2 and WO 2017/179017 A1.

As used herein, a “chimeric dengue serotype 2/1 strain” or “DENV-2/1 chimera” or “TDV-1” refers to a dengue virus chimeric construct which comprises parts from both DENV-2 and DENV-1. In particular, in the chimeric dengue serotype 2/1 strain the prM and E proteins from DENV-1 replace the prM and E proteins from DENV-2. In one embodiment, the chimeric dengue serotype 2/1 strain has the nucleotide sequence according to SEQ ID No. 1 and/or the amino acid sequence according to SEQ ID No. 2.

As used herein, a “chimeric dengue serotype 2/3 strain” or “DENV-2/3 chimera” or “TDV-3” refers to a dengue virus chimeric construct which comprises parts from both DENV-2 and DENV-3. In particular, in the chimeric dengue serotype 2/3 strain the prM and E proteins from DENV-3 replace the prM and E proteins from DENV-2. In one embodiment, TDV-3 has the nucleotide sequence according to SEQ ID No. 5 and/or the amino acid sequence according to SEQ ID No. 6.

As used herein, a “chimeric dengue serotype 2/4 strain” or “DENV-2/4 chimera” or “TDV-4” refers to a dengue virus chimeric construct which comprises parts from both DENV-2 and DENV-4. In particular, in the chimeric dengue serotype 2/4 strain the prM and E proteins from DENV-4 replace the prM and E proteins from DENV-2 as detailed below. In one embodiment, TDV-4 has the nucleotide sequence according to SEQ ID No. 7 and/or the amino acid sequence according to SEQ ID No. 8.

In the method of the present invention the sample comprising infectious viral particles is mixed with a composition comprising one or more of a non-ionic surfactant, a sugar and a protein. In one embodiment of the method of the present invention the sample comprising infectious viral particles is mixed with a composition comprising a non-ionic surfactant. In one embodiment of the method of the present invention the sample comprising infectious viral particles is mixed with a composition comprising a non-ionic surfactant, a sugar and a protein.

The term “non-ionic surfactant” means a surfactant that contains neither positively nor negatively charged (i.e. ionic) functional groups. In contrast to anionic and cationic surfactants, non-ionic surfactants do not ionize in solution. Non-ionic surfactants may be selected from block copolymers, sorbitan esters, ethoxylated or propoxylated sorbitan esters, alkyl-polyglycosides (APG), alkoxylated mono- or di-alkylamines, fatty acid monoethanolamides (FAMA), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), and combinations thereof.

The non-ionic surfactant is preferably a high molecular weight non-ionic surfactant. “High molecular weight” means a molecular weight of 1500 or more. In one embodiment, the non-ionic surfactant is a non-ionic triblock copolymer. In one embodiment, the surfactant is a non-ionic, hydrophilic, polyoxyethylene-polyoxypropylene block copolymer (or EO-PO block copolymer). The EO-PO block copolymers can include blocks of polyethylene oxide (—CH2CH2O-designated EO) and polypropylene oxide (—CH2CHCH3O— designated PO). The PO block can be flanked by two EO blocks in a EOx-POy-Eox arrangement. Since the PO component is hydrophilic and the EO component is hydrophobic, the overall hydrophilicity, molecular weight and the surfactant properties of the copolymer can be adjusted by varying x and y in the EOx-POy-Eox block structure. In aqueous solutions, the EO-PO block copolymers will self-assemble into micelles with a PO core and a corona of hydrophilic EO groups.

In one embodiment, the non-ionic surfactant is a poloxamer. Poloxamers are non-ionic triblock copolymers composed of a central hydrophobic chain of poly(propyleneoxide) flanked by two hydrophilic chains of poly(ethylene oxide). The length of the polymer blocks can be customized, leading to different poloxamers with slightly different properties.

In one embodiment, the non-ionic surfactant is poloxamer 407 or poloxamer 403. Preferably, the non-ionic surfactant is poloxamer 407. Poloxamer 407 is a hydrophilic non-ionic surfactant which consists of a triblock copolymer consisting of a central propylene glycol block with about 56 repeat units and two flanking hydrophilic polyethylene glycol blocks each comprising about 101 repeat units. Poloxamer 407 is also known by its trade names Pluronic F127 and Synperonic PE/F127.

The concentration of the non-ionic surfactant in the mixture with the sample comprising infectious viral particles and host cell DNA is 0.05% to 0.5% (w/v). Preferably, the concentration of the non-ionic surfactant in the mixture with the sample comprising infectious viral particles and host cell DNA is 0.08% to 0.4% (w/v), more preferably, the concentration of the non-ionic surfactant in the mixture with the sample comprising infectious viral particles and host cell DNA is 0.1% to 0.3% (w/v) and most preferably the concentration of the non-ionic surfactant in the mixture with the sample comprising infectious viral particles and host cell DNA is 0.2% (w/v).

The concentration of the non-ionic triblock copolymer in the mixture with the sample comprising infectious viral particles and host cell DNA is 0.05% to 0.5% (w/v). Preferably, the concentration of the non-ionic triblock copolymer in the mixture with the sample comprising infectious viral particles and host cell DNA is 0.08% to 0.4% (w/v), more preferably, the concentration of the non-ionic triblock copolymer in the mixture with the sample comprising infectious viral particles and host cell DNA is 0.1% to 0.3% (w/v) and most preferably the concentration of the non-ionic triblock copolymer in the mixture with the sample comprising infectious viral particles and host cell DNA is 0.2% (w/v).

The concentration of poloxamer 407 in the mixture with the sample comprising infectious viral particles and host cell DNA is 0.05% to 0.5% (w/v). Preferably, the concentration of poloxamer 407 in the mixture with the sample comprising infectious viral particles and host cell DNA is 0.08% to 0.4% (w/v), more preferably, the concentration of poloxamer 407 in the mixture with the sample comprising infectious viral particles and host cell DNA is 0.1% to 0.3% (w/v) and most preferably the concentration of poloxamer 407 in the mixture with the sample comprising infectious viral particles and host cell DNA is 0.2% (w/v).

As used herein, the term “sugar” includes monosaccharides, (e.g. glucose, galactose, ribose, mannose, rhamnose, talose, xylose, or allose arabinose), disaccharides (e.g. trehalose, sucrose, maltose, isomaltose, cellibiose, gentiobiose, laminaribose, xylobiose, mannobiose, lactose, or fructose), trisaccharides (e.g. acarbose, raffinose, melizitose, panose, or cellotriose) and sugar polymers (e.g. dextran, xanthan, pullulan, cyclodextrins, amylose, amylopectin, starch, celloologosaccharides, cellulose, maltooligosaccharides, glycogen, chitosan, or chitin). In addition, the term “sugar” as used herein also includes sugar alcohols such as mannitol, sorbitol, arabitol, erythritol, maltitol, xylitol, glycitol, glycol, polyglycitol, polyethylene glycol, polypropylene glycol, and glycerol.

In one embodiment, the sugar is a non-reducing sugar. Non-reducing sugars do not contain an aldehyde or ketone group which is capable of being oxidized. Examples of non-reducing sugars include sucrose and trehalose. In one embodiment, the sugar is trehalose. In one embodiment, the sugar is trehalose dihydrate.

The concentration of the sugar in the mixture with the sample comprising infectious viral particles and host cell DNA is 1% to 7.5% (w/v). Preferably, the concentration of the sugar in the mixture with the sample comprising infectious viral particles and host cell DNA is 1.5% to 6% (w/v), more preferably, the concentration of the sugar in the mixture with the sample comprising infectious viral particles and host cell DNA is 2% to 5% (w/v) and most preferably the concentration of the sugar in the mixture with the sample comprising infectious viral particles and host cell DNA is 3% (w/v).

The concentration of the non-reducing sugar in the mixture with the sample comprising infectious viral particles and host cell DNA is 1% to 7.5% (w/v). Preferably, the concentration of the non-reducing sugar in the mixture with the sample comprising infectious viral particles and host cell DNA is 1.5% to 6% (w/v), more preferably, the concentration of the non-reducing sugar in the mixture with the sample comprising infectious viral particles and host cell DNA is 2% to 5% (w/v) and most preferably the concentration of the non-reducing sugar in the mixture with the sample comprising infectious viral particles and host cell DNA is 3% (w/v).

The concentration of trehalose dihydrate in the mixture with the sample comprising infectious viral particles and host cell DNA is 1% to 7.5% (w/v). Preferably, the concentration of trehalose dihydrate in the mixture with the sample comprising infectious viral particles and host cell DNA is 1.5% to 6% (w/v), more preferably, the concentration of trehalose dihydrate in the mixture with the sample comprising infectious viral particles and host cell DNA is 2% to 5% (w/v) and most preferably the concentration of trehalose dihydrate in the mixture with the sample comprising infectious viral particles and host cell DNA is 3% (w/v).

In one embodiment, the concentration of the non-ionic surfactant in the mixture with the sample comprising infectious viral particles and host cell DNA is 0.05% to 0.5% (w/v) and the concentration of the sugar in the mixture with the sample comprising infectious viral particles and host cell DNA is 1% to 7.5% (w/v). Preferably, the concentration of the non-ionic surfactant in the mixture with the sample comprising infectious viral particles and host cell DNA is 0.08% to 0.4% (w/v) and the concentration of the sugar in the mixture with the sample comprising infectious viral particles and host cell DNA is 1.5% to 6% (w/v). More preferably, the concentration of the non-ionic surfactant in the mixture with the sample comprising infectious viral particles and host cell DNA is 0.1% to 0.3% (w/v) and the concentration of the sugar in the mixture with the sample comprising infectious viral particles and host cell DNA is 2% to 5% (w/v). Most preferably the concentration of the non-ionic surfactant in the mixture with the sample comprising infectious viral particles and host cell DNA is 0.2% (w/v) and the concentration of the sugar in the mixture with the sample comprising infectious viral particles and host cell DNA is 3% (w/v).

In one embodiment, the concentration of the non-ionic triblock copolymer in the mixture with the sample comprising infectious viral particles and host cell DNA is 0.05% to 0.5% (w/v) and the concentration of the non-reducing sugar in the mixture with the sample comprising infectious viral particles and host cell DNA is 1% to 7.5% (w/v). Preferably, the concentration of the non-ionic triblock copolymer in the mixture with the sample comprising infectious viral particles and host cell DNA is 0.08% to 0.4% (w/v) and the concentration of the non-reducing sugar in the mixture with the sample comprising infectious viral particles and host cell DNA is 1.5% to 6% (w/v). More preferably, the concentration of the non-ionic triblock copolymer in the mixture with the sample comprising infectious viral particles and host cell DNA is 0.1% to 0.3% (w/v) and the concentration of the non-reducing sugar in the mixture with the sample comprising infectious viral particles and host cell DNA is 2% to 5% (w/v). Most preferably the concentration of the non-ionic triblock copolymer in the mixture with the sample comprising infectious viral particles and host cell DNA is 0.2% (w/v) and the concentration of the non-reducing sugar in the mixture with the sample comprising infectious viral particles and host cell DNA is 3% (w/v).

In one embodiment, the concentration of poloxamer 407 in the mixture with the sample comprising infectious viral particles and host cell DNA is 0.05% to 0.5% (w/v) and the concentration of trehalose dihydrate in the mixture with the sample comprising infectious viral particles and host cell DNA is 1% to 7.5% (w/v). Preferably, the concentration of poloxamer 407 in the mixture with the sample comprising infectious viral particles and host cell DNA is 0.08% to 0.4% (w/v) and the concentration of trehalose dihydrate in the mixture with the sample comprising infectious viral particles and host cell DNA is 1.5% to 6% (w/v). More preferably, the concentration of poloxamer 407 in the mixture with the sample comprising infectious viral particles and host cell DNA is 0.1% to 0.3% (w/v) and the concentration of trehalose dihydrate in the mixture with the sample comprising infectious viral particles and host cell DNA is 2% to 5% (w/v). Most preferably the concentration of poloxamer 407 in the mixture with the sample comprising infectious viral particles and host cell DNA is 0.2% (w/v) and the concentration of trehalose dihydrate in the mixture with the sample comprising infectious viral particles and host cell DNA is 3% (w/v).

The protein which may be present in the mixture with the sample comprising infectious viral particles and host cell DNA may be any protein which is essentially inert and does not react with the infectious viral particle. In particular, the protein does not affect the structure or infectivity of the infectious viral particle. In one embodiment, the protein is not an enzyme or an antibody. In one embodiment, the protein is a structural protein or a serum protein. In one embodiment, the protein is selected from an albumin, collagen, hydrolyzed collagen, gelatin and hydrolyzed gelatin.

In one embodiment, the protein is an albumin. The albumins are a family of globular non-glycosylated proteins which are inter alia present in the blood of a vertebrate. They are water-soluble, moderately soluble in concentrated salt solutions, and experience heat denaturation. Suitable albumins for use in the method of the present invention include mammalian serum albumins such as human serum albumin and bovine serum albumin or lactalbumin.

In one embodiment, the protein is human serum albumin. Serum albumin is one of the most common proteins in vertebrate blood and has multiple functions. The protein is 585 amino acids with a molecular weight of 66,500 D. Human serum albumin is not glycosylated and has a single free thiol group. The human serum albumin may be recombinant human serum albumin or it may be human serum albumin purified from human serum. Preferably, human serum albumin purified from human serum is used.

The concentration of the protein in the mixture with the sample comprising infectious viral particles and host cell DNA is 0.005% to 0.05% (w/v). Preferably, the concentration of the protein in the mixture with the sample comprising infectious viral particles and host cell DNA is 0.01% to 0.04% (w/v), more preferably, the concentration of the protein in the mixture with the sample comprising infectious viral particles and host cell DNA is 0.015% to 0.03% (w/v) and most preferably the concentration of the protein in the mixture with the sample comprising infectious viral particles and host cell DNA is 0.02% (w/v).

The concentration of the albumin in the mixture with the sample comprising infectious viral particles and host cell DNA is 0.005% to 0.05% (w/v). Preferably, the concentration of the albumin in the mixture with the sample comprising infectious viral particles and host cell DNA is 0.01% to 0.04% (w/v), more preferably, the concentration of the albumin in the mixture with the sample comprising infectious viral particles and host cell DNA is 0.015% to 0.03% (w/v) and most preferably the concentration of the albumin in the mixture with the sample comprising infectious viral particles and host cell DNA is 0.02% (w/v).

The concentration of human serum albumin in the mixture with the sample comprising infectious viral particles and host cell DNA is 0.005% to 0.05% (w/v). Preferably, the concentration of human serum albumin in the mixture with the sample comprising infectious viral particles and host cell DNA is 0.01% to 0.04% (w/v), more preferably, the concentration of human serum albumin in the mixture with the sample comprising infectious viral particles and host cell DNA is 0.015% to 0.03% (w/v) and most preferably the concentration of human serum albumin in the mixture with the sample comprising infectious viral particles and host cell DNA is 0.02% (w/v).

In one embodiment, the concentration of the non-ionic surfactant in the mixture with the sample comprising infectious viral particles and host cell DNA is 0.05% to 0.5% (w/v) and the concentration of the protein in the mixture with the sample comprising infectious viral particles and host cell DNA is 0.005% to 0.05% (w/v). Preferably, the concentration of the non-ionic surfactant in the mixture with the sample comprising infectious viral particles and host cell DNA is 0.08% to 0.4% (w/v) and the concentration of the protein in the mixture with the sample comprising infectious viral particles and host cell DNA is 0.01% to 0.04% (w/v). More preferably, the concentration of the non-ionic surfactant in the mixture with the sample comprising infectious viral particles and host cell DNA is 0.1% to 0.3% (w/v) and the concentration of the protein in the mixture with the sample comprising infectious viral particles and host cell DNA is 0.015% to 0.03% (w/v). Most preferably, the concentration of the non-ionic surfactant in the mixture with the sample comprising infectious viral particles and host cell DNA is 0.2% (w/v) and the concentration of the protein in the mixture with the sample comprising infectious viral particles and host cell DNA is 0.02% (w/v).

In one embodiment, the concentration of the non-ionic triblock copolymer in the mixture with the sample comprising infectious viral particles and host cell DNA is 0.05% to 0.5% (w/v) and the concentration of the albumin in the mixture with the sample comprising infectious viral particles and host cell DNA is 0.005% to 0.05% (w/v). Preferably, the concentration of the non-ionic triblock copolymer in the mixture with the sample comprising infectious viral particles and host cell DNA is 0.08% to 0.4% (w/v) and the concentration of the albumin in the mixture with the sample comprising infectious viral particles and host cell DNA is 0.01% to 0.04% (w/v). More preferably, the concentration of the non-ionic triblock copolymer in the mixture with the sample comprising infectious viral particles and host cell DNA is 0.1% to 0.3% (w/v) and the concentration of the albumin in the mixture with the sample comprising infectious viral particles and host cell DNA is 0.015% to 0.03% (w/v). Most preferably, the concentration of the non-ionic triblock copolymer in the mixture with the sample comprising infectious viral particles and host cell DNA is 0.2% (w/v) and the concentration of the albumin in the mixture with the sample comprising infectious viral particles and host cell DNA is 0.02% (w/v).

In one embodiment, the concentration of poloxamer 407 in the mixture with the sample comprising infectious viral particles and host cell DNA is 0.05% to 0.5% (w/v) and the concentration of human serum albumin in the mixture with the sample comprising infectious viral particles and host cell DNA is 0.005% to 0.05% (w/v). Preferably, the concentration of poloxamer 407 in the mixture with the sample comprising infectious viral particles and host cell DNA is 0.08% to 0.4% (w/v) and the concentration of human serum albumin in the mixture with the sample comprising infectious viral particles and host cell DNA is 0.01% to 0.04% (w/v). More preferably, the concentration of poloxamer 407 in the mixture with the sample comprising infectious viral particles and host cell DNA is 0.1% to 0.3% (w/v) and the concentration of human serum albumin in the mixture with the sample comprising infectious viral particles and host cell DNA is 0.015% to 0.03% (w/v). Most preferably, the concentration of poloxamer 407 in the mixture with the sample comprising infectious viral particles and host cell DNA is 0.2% (w/v) and the concentration of human serum albumin in the mixture with the sample comprising infectious viral particles and host cell DNA is 0.02% (w/v).

In one embodiment, the concentration of the sugar in the mixture with the sample comprising infectious viral particles and host cell DNA is 1% to 7.5% (w/v) and the concentration of the protein in the mixture with the sample comprising infectious viral particles and host cell DNA is 0.005% to 0.05% (w/v). Preferably, the concentration of the sugar in the mixture with the sample comprising infectious viral particles and host cell DNA is 1.5% to 6% (w/v) and the concentration of the protein in the mixture with the sample comprising infectious viral particles and host cell DNA is 0.01% to 0.04% (w/v). More preferably, the concentration of the sugar in the mixture with the sample comprising infectious viral particles and host cell DNA is 2% to 5% (w/v) and the concentration of the protein in the mixture with the sample comprising infectious viral particles and host cell DNA is 0.015% to 0.03% (w/v). Most preferably, the concentration of the sugar in the mixture with the sample comprising infectious viral particles and host cell DNA is 3% (w/v) and the concentration of the protein in the mixture with the sample comprising infectious viral particles and host cell DNA is 0.02% (w/v).

In one embodiment, the concentration of the non-reducing sugar in the mixture with the sample comprising infectious viral particles and host cell DNA is 1% to 7.5% (w/v) and the concentration of the albumin in the mixture with the sample comprising infectious viral particles and host cell DNA is 0.005% to 0.05% (w/v). Preferably, the concentration of the non-reducing sugar in the mixture with the sample comprising infectious viral particles and host cell DNA is 1.5% to 6% (w/v) and the concentration of the albumin in the mixture with the sample comprising infectious viral particles and host cell DNA is 0.01% to 0.04% (w/v). More preferably, the concentration of the non-reducing sugar in the mixture with the sample comprising infectious viral particles and host cell DNA is 2% to 5% (w/v) and the concentration of the albumin in the mixture with the sample comprising infectious viral particles and host cell DNA is 0.015% to 0.03% (w/v). Most preferably, the concentration of the non-reducing sugar in the mixture with the sample comprising infectious viral particles and host cell DNA is 3% (w/v) and the concentration of the albumin in the mixture with the sample comprising infectious viral particles and host cell DNA is 0.02% (w/v).

In one embodiment, the concentration of trehalose dihydrate in the mixture with the sample comprising infectious viral particles and host cell DNA is 1% to 7.5% (w/v) and the concentration of human serum albumin in the mixture with the sample comprising infectious viral particles and host cell DNA is 0.005% to 0.05% (w/v). Preferably, the concentration of trehalose dihydrate in the mixture with the sample comprising infectious viral particles and host cell DNA is 1.5% to 6% (w/v) and the concentration of human serum albumin in the mixture with the sample comprising infectious viral particles and host cell DNA is 0.01% to 0.04% (w/v). More preferably, the concentration of trehalose dihydrate in the mixture with the sample comprising infectious viral particles and host cell DNA is 2% to 5% (w/v) and the concentration of human serum albumin in the mixture with the sample comprising infectious viral particles and host cell DNA is 0.015% to 0.03% (w/v). Most preferably, the concentration of trehalose dihydrate in the mixture with the sample comprising infectious viral particles and host cell DNA is 3% (w/v) and the concentration of human serum albumin in the mixture with the sample comprising infectious viral particles and host cell DNA is 0.02% (w/v).

In one embodiment, the concentration of the non-ionic surfactant in the mixture with the sample comprising infectious viral particles and host cell DNA is 0.05% to 0.5% (w/v), the concentration of the sugar in the mixture with the sample comprising infectious viral particles and host cell DNA is 1% to 7.5% (w/v) and the concentration of the protein in the mixture with the sample comprising infectious viral particles and host cell DNA is 0.005% to 0.05% (w/v). Preferably, the concentration of the non-ionic surfactant in the mixture with the sample comprising infectious viral particles and host cell DNA is 0.08% to 0.4% (w/v), the concentration of the sugar in the mixture with the sample comprising infectious viral particles and host cell DNA is 1.5% to 6% (w/v) and the concentration of the protein in the mixture with the sample comprising infectious viral particles and host cell DNA is 0.01% to 0.04% (w/v). More preferably, the concentration of the non-ionic surfactant in the mixture with the sample comprising infectious viral particles and host cell DNA is 0.1% to 0.3% (w/v), the concentration of the sugar in the mixture with the sample comprising infectious viral particles and host cell DNA is 2% to 5% (w/v) and the concentration of the protein in the mixture with the sample comprising infectious viral particles and host cell DNA is 0.015% to 0.03% (w/v). Most preferably, the concentration of the non-ionic surfactant in the mixture with the sample comprising infectious viral particles and host cell DNA is 0.2% (w/v), the concentration of the sugar in the mixture with the sample comprising infectious viral particles and host cell DNA is 3% (w/v) and the concentration of the protein in the mixture with the sample comprising infectious viral particles and host cell DNA is 0.02% (w/v).

In one embodiment, the concentration of the non-ionic triblock copolymer in the mixture with the sample comprising infectious viral particles and host cell DNA is 0.05% to 0.5% (w/v), the concentration of the non-reducing sugar in the mixture with the sample comprising infectious viral particles and host cell DNA is 1% to 7.5% (w/v) and the concentration of the albumin in the mixture with the sample comprising infectious viral particles and host cell DNA is 0.005% to 0.05% (w/v). Preferably, the concentration of the non-ionic triblock copolymer in the mixture with the sample comprising infectious viral particles and host cell DNA is 0.08% to 0.4% (w/v), the concentration of the non-reducing sugar in the mixture with the sample comprising infectious viral particles and host cell DNA is 1.5% to 6% (w/v) and the concentration of the albumin in the mixture with the sample comprising infectious viral particles and host cell DNA is 0.01% to 0.04% (w/v). More preferably, the concentration of the non-ionic triblock copolymer in the mixture with the sample comprising infectious viral particles and host cell DNA is 0.1% to 0.3% (w/v), the concentration of the non-reducing sugar in the mixture with the sample comprising infectious viral particles and host cell DNA is 2% to 5% (w/v) and the concentration of the albumin in the mixture with the sample comprising infectious viral particles and host cell DNA is 0.015% to 0.03% (w/v). Most preferably, the concentration of the non-ionic triblock copolymer in the mixture with the sample comprising infectious viral particles and host cell DNA is 0.2% (w/v), the concentration of the non-reducing sugar in the mixture with the sample comprising infectious viral particles and host cell DNA is 3% (w/v) and the concentration of the albumin in the mixture with the sample comprising infectious viral particles and host cell DNA is 0.02% (w/v).

In one embodiment, the concentration of poloxamer 407 in the mixture with the sample comprising infectious viral particles and host cell DNA is 0.05% to 0.5% (w/v), the concentration of trehalose dihydrate in the mixture with the sample comprising infectious viral particles and host cell DNA is 1% to 7.5% (w/v) and the concentration of human serum albumin in the mixture with the sample comprising infectious viral particles and host cell DNA is 0.005% to 0.05% (w/v). Preferably, the concentration of poloxamer 407 in the mixture with the sample comprising infectious viral particles and host cell DNA is 0.08% to 0.4% (w/v), the concentration of trehalose dihydrate in the mixture with the sample comprising infectious viral particles and host cell DNA is 1.5% to 6% (w/v) and the concentration of human serum albumin in the mixture with the sample comprising infectious viral particles and host cell DNA is 0.01% to 0.04% (w/v). More preferably, the concentration of poloxamer 407 in the mixture with the sample comprising infectious viral particles and host cell DNA is 0.1% to 0.3% (w/v), the concentration of trehalose dihydrate in the mixture with the sample comprising infectious viral particles and host cell DNA is 2% to 5% (w/v) and the concentration of human serum albumin in the mixture with the sample comprising infectious viral particles and host cell DNA is 0.015% to 0.03% (w/v). Most preferably, the concentration of poloxamer 407 in the mixture with the sample comprising infectious viral particles and host cell DNA is 0.2% (w/v), the concentration of trehalose dihydrate in the mixture with the sample comprising infectious viral particles and host cell DNA is 3% (w/v) and the concentration of human serum albumin in the mixture with the sample comprising infectious viral particles and host cell DNA is 0.02% (w/v).

Anion exchange chromatography relies on charge-charge interactions between components in a sample and the charges of the immobilized functional group. In anion exchange chromatography, the binding ions present in the sample are negative, and the immobilized functional group is positive. Since most viruses are negatively charged at physiological pH, they bind to the immobilized positive groups. Commonly used anion exchange functional groups are Q-resin, a quaternary amine, and DEAE resin (diethylaminoethane). However, in general the anion exchange chromatography step can be performed with all common commercially available anion exchange resins or membranes.

Typical strong anion exchange groups that can be used for the purpose of the invention comprise functional groups such as: quaternary aminoethyl (QAE) moieties, primary amine (PA), quaternary ammonium (Q) moieties and trimethylammoniumethyl (TMAE) groups.

Resins having quaternary aminoethyl (QAE) moieties include, e.g., Toyopearl QAE (available from Tosoh Bioscience, Germany), Selectacel QAE (a quaternary aminoethyl derivative of cellulose, available from Polysciences Inc., Pennsylvania USA) and others. Resins having quaternary ammonium (Q) moieties include, e.g., Sartobind® Q (available from Sartorius, Germany), Mustang® Q Acrodisc (available from Pall, Germany), Q Sepharose XL, Q Sepharose FF, Q Sepharose HP, Resource Q (available from GE Healthcare, Germany), Macro Prep High Q (Bio-Rad, California, USA), Toyopearl Super Q (available from Tosoh Bioscience, Germany) and UNOsphere Q (available from Bio-Rad, California, USA). Resins having trimethylammoniumethyl (TMAE) groups include, e.g., Fractogel EMD TMAE (available from Merck, Germany). Resins having primary amine groups include Sartobind STIC primary amine resins (available from Sartorius, Germany).

In the method of the present invention the anion exchange chromatography step is preferably performed with an anion exchange chromatography membrane having quaternary ammonium groups. The membrane base material is selected from stabilized reinforced cellulose and polyethersulfone. Preferably, the membrane base material is stabilized reinforced cellulose. Preferably, the membrane base material is stabilized reinforced cellulose and the functional group is a quaternary ammonium group. Preferably, the anion exchange chromatography step does not involve the use of a monolithic support. The nominal pore size of the membrane is 0.5 μm to 5 μM, preferably it is greater than 3 μm. The membrane area is 20 to 50 cm², preferably 25 to 45 cm², more preferably 30 to 40 cm² and most preferably 36 cm². The flow rate is 1 to 50 all/min, preferably 10 to 40 all/min and more preferably 30 all/min. Most preferably, the anion exchange chromatography is performed using a Sartobind® Q SingleStep membrane.

Anion exchange chromatography may be performed in bind-and-elute or in flow-through mode. In the bind-and-elute mode the substance to be purified binds to the anion exchange groups while the impurities do not bind. The substance to be purified can be eluted from the anion exchange groups by changing one or more chromatography conditions such as the salt condition in the buffer or the pH of the buffer. In the flow-through mode the impurities bind to the anion exchange groups while the substance to be purified does not bind, but can be collected directly from the flow-through. In the present invention the anion exchange chromatography is preferably performed in flow-through mode. This means that in the present invention the host cell DNA binds to the anion exchange groups, preferably the quaternary ammonium groups, and the infectious viral particles are present in the flow-through.

The method of the present invention minimizes the loss of viral titer during the purification process. In one embodiment, less than 5% of the viral titer are lost during the anion exchange chromatography process. Preferably, less than 4% of the viral titer are lost during the anion exchange chromatography process. More preferably, less than 3% of the viral titer are lost during the anion exchange chromatography process. Most preferably, less than 2% or 1.5% of the viral titer are lost during the anion exchange chromatography process.

The viral titer can be determined by any method known in the art. In one embodiment, the viral titer is determined by an immuno-focus assay known in the art. In an immuno-focus assay serial dilutions of virus are applied to monolayers of adherent cells, such as Vero cells. After a period of time which allows infectious viruses to bind to the cells and to be taken up by the cells, an overlay containing thickening agents, such as agarose or carboxymethylcellulose, is added to prevent diffusion of viruses so that progeny viruses can only infect cells adjacent to the original infected cells. After a period of incubation to allow viral replication, cells are fixed and stained using monoclonal antibodies directed to viral proteins and a secondary antibody such as an antibody labeled with alkaline phosphatase. The foci are stained by adding a suitable substrate for the enzyme attached to the secondary antibody, such as 5-bromo-4-chloro-3-indolyl-phosphate/nitro blue tetrazolium phosphatase substrate. The number of plaques on the plate corresponds to the plaque forming units of the virus in the solutions applied to the cells. For example, a concentration of 1.000 pfu/μl indicates that 1 μl of the solution applied to the cells contains enough viruses to produce 1.000 plaques in a cell monolayer.

In some prior art methods the host cell DNA is removed by treatment with an enzyme capable of degrading DNA such as a nuclease, for example an endonuclease. An endonuclease commonly used for host cell DNA removal is benzonase. Benzonase is a modified form of the extracellular endonuclease from Serratia marcescens which is available e.g. from Sigma Aldrich or Merck Millipore. Since the method of the present invention efficiently removes host cell DNA without enzymatic treatment, the method of the present invention does not include a step of treating the sample comprising infectious viral particles and host cell DNA with a nuclease. In particular, the method of the present invention does not include a step of treating the sample comprising infectious viral particles and host cell DNA with benzonase. In one embodiment, the method of the present invention does not include any step of endonuclease treatment.

As discussed before, the infectious viral particles are prepared in host cells which are capable of being infected by the infectious viruses and replicate the viruses therein when they are maintained under suitable conditions. Typically, mammalian host cells are used. Suitable mammalian host cells include, but are not limited to, Vero, MDCK, BHK, HEK-293 (293), RD, HT-1080, A549, Cos-7, ARPE-19 and MRC-5 cells. Preferably, Vero cells are used for preparing the infectious viral particles. More preferably, the Vero cells are derived from WHO reference cell bank 10-87.

The host cells used for producing the infectious viral particles are cultured under suitable conditions which allow the host cells to grow and the virus to replicate within the host cells. In one embodiment, the host cells are cultured before infection in DMEM with 10% fetal bovine serum (FBS) and without phenol red. In one embodiment, the host cells are cultured before infection at a temperature between 36° C. and 38° C. In one embodiment, the host cells are cultured before infection in DMEM with 10% fetal bovine serum (FBS) and without phenol red and at a temperature between 36° C. and 38° C.

The medium used for infecting the host cells with viruses may be different from the medium used for culturing the host cells before the infection. In one embodiment, the medium used for infecting the host cells may be DMEM with 0.1% F127.

The medium used for culturing the host cells after infection with the viruses may be different from the medium used for culturing the host cells before the infection. In one embodiment, the medium used for culturing the host cells after infection may be DMEM with 0.1% F127.

After the host cells have been cultured for a certain period, the cell supernatant containing the infectious viral particles may be harvested. In one embodiment, the host cells are cultured for four to ten days, before the cell supernatant containing the infectious viral particles is harvested. In one embodiment, the cell supernatant containing the infectious viral particles is harvested on day 5, day 6, day 7, day 8, day 9 or day 10 of the culture. In one embodiment, the cell supernatant containing the infectious viral particles is harvested on day 5, day 6, day 7, day 8, day 9 and day 10 of the culture and after harvesting the cell supernatant containing the infectious viral particles on day 5, day 6, day 7, day 8 and day 9 fresh medium is added to the host cells to allow the cells to continue virus production until the next harvest. The cell supernatant containing the infectious viral particles obtained on each day may be purified separately or the cell supernatant containing the infectious viral particles obtained on each day may be mixed with the cell supernatant containing the infectious viral particles obtained on the other days and then purified.

After the host cell culture supernatant has been harvested, it is subjected to one or more depth filtration steps. Depth filtration uses a porous filtration medium to separate particles and solids from a liquid. The filter media which may be used in depth filtration include, but are not limited to, cellulose acetate, polypropylene, cellulose acetate protected by glassfiber fleece and polyethersulfone. In one embodiment, the filter medium used in depth filtration is polyethersulfone. The filter used in depth filtration may have a pore size of 0.1 μm to 1 μm, preferably of 0.2 μm to 0.8 μm and more preferably of 0.2 μm to 0.45 μm. In one embodiment, the filter is a heterogenous double layer of polyethersulfone with pore sizes of 0.2 μm and 0.45 μm. Such a filter is available as Sartopore 2.

After the depth filtration step the filtrate which contains the harvested infectious viral particles is mixed with a composition comprising one or more of a surfactant, a sugar and a protein, thereby providing a mixture. In one embodiment, the filtrate is mixed with a composition comprising a surfactant, a sugar and a protein, thereby providing a mixture. The surfactant, the sugar and the protein and their concentrations in the mixture are defined as discussed above. The composition comprising a surfactant, a sugar and a protein is diluted by mixing it with the filtrate. The composition comprising a surfactant, a sugar and a protein is diluted two- to ten-fold, preferably three- to eight-fold, more preferably four- to six-fold and most preferably five-fold. The final concentration of the surfactant, sugar and protein after dilution is provided above.

After the anion exchange chromatography has been performed, the sample containing the infectious viral particles is subjected to one or more tangential flow filtration (TFF) steps, preferably one TFF step. The TFF step serves to concentrate the sample to ensure there is sufficient potency and to remove small molecular weight impurities. In TFF the majority of the feed travels tangentially across the surface of the filter and not into the filter.

Suitable filter materials for TFF include, but are not limited to, cross-linked cellulose and polyethersulfone. Preferably, a cross-linked cellulose based polymer is used which is available from Sartorius under the name Hydrosart. The cut-off size of the filter material used in tangential flow filtration determines whether a certain compound is present in the filtrate or the retentate. The cut-off size may be smaller than ⅓ to ⅕ of the size of the virus to be purified. In one embodiment, the cut-off size is between 50 and 300 kDa. Preferably, the cut-off size is 100 kDa. In one embodiment, a cross-linked cellulose based polymer filter with a cut-off size of 100 kDa is used in the TFF step. Hence, the infectious viral particles are present in the retentate.

The TFF process can be characterized by the flow rate and the transmembrane pressure. The flow rate is proportional to the transmembrane pressure and inverse proportional to the resistance of the membrane and the filter cake. The flow rate is given as the feed flow rate per unit area of membrane which is liter per meter squared per hour (LMH). In the TFF step of the present invention the flow rate is between 100 and 500 LMH, preferably between 120 and 400 LMH and more preferably between 150 and 300 LMH. Most preferably, the flow rate is 300 LMH. Hence, the TFF step in the method of the present invention is preferably performed using a cross-linked cellulose based polymer filter with a cut-off size of 100 kDa and a flow rate of 300 LMH. The transmembrane pressure (TMP) is the pressure difference between two sides of the membrane. The TMP may be 0.2 to 0.5 bar, preferably, 0.25 to 0.45 bar, more preferably 0.35 to 0.45 bar and most preferably 0.35 to 0.40 bar. Hence, the TFF step in the method of the present invention is preferably performed using a cross-linked cellulose based polymer filter with a cut-off size of 100 kDa, a flow rate of 300 LMH and a TMP of 0.35 to 0.40 bar.

Two basic filter configurations are generally used for TFF: cartridge filters and cassette filters. In cartridge filters (often called hollow fiber filters), the membrane forms a set of parallel hollow fibers. The feed stream passes through the lumen of the fibers and the permeate is collected from outside the fibers. In cassette filters, several flat sheets of membrane are held apart from each other and from the cassette housing by support screens. The feed stream passes into the space between two sheets and permeate is collected from the opposite side of the sheets. In the TFF step in the method of the present invention preferably a cassette filter is used. In one embodiment of the present invention the TFF step does not use hollow fiber filters.

Preferably, the TFF step excludes diafiltration. Also preferably, the filter membrane is flushed with buffer one to five times, preferably two times. The buffer used for flushing comprises a non-ionic surfactant, a non-reducing sugar and an albumin. Preferably, the buffer used for flushing comprises poloxamer 407, trehalose dihydrate and human serum albumin. The buffer used for flushing comprises 0.5 to 3% of a non-ionic surfactant, 20 to 40% of a non-reducing sugar and 0.05 to 0.3% of an albumin. The buffer used for flushing comprises 0.5 to 3% of poloxamer 407, 20 to 40% of trehalose dihydrate and 0.05 to 0.3% of human serum albumin. The buffer used for flushing comprises 1% of a non-ionic surfactant, 27% of a non-reducing sugar and 0.1% of an albumin. The buffer used for flushing comprises 1% of poloxamer 407, 27% of trehalose dihydrate and 0.1% of human serum albumin.

In the TFF step the solution containing the infectious viral particles is concentrated two- to ten-fold, preferably three- to eight-fold, more preferably four- to six-fold and most preferably five-fold.

After the TFF step has been performed, the purified infectious viral particles are recovered from the retentate. In the retentate, the purified infectious viral particles are present in solution. The solution containing the purified infectious viral particles may be filtered one or more times. The solution containing the infectious viral particles may be frozen, before it is used to produce the drug product.

In one embodiment, the method for purifying infectious viral particles further comprises a step (f) of preparing a drug product. The drug product may contain more than one type of infectious viral particles. For example, if the infectious viral particles are infectious dengue virus particles, the drug product may contain more than one serotype of the dengue virus. Preferably, the drug product contains infectious viral particles of all four serotypes of the dengue virus. Hence, in this case step (f) of preparing a drug product may involve the mixing of infectious viral particles of four dengue serotypes. Step (f) of preparing a drug product may also include the preparation of a dried form of the drug product, e.g. by lyophilizing or spray-drying. In one embodiment step (f) of preparing a drug product includes the mixing of infectious viral particles of four dengue serotypes and lyophilizing the mixture. Methods for lyophilizing a mixture containing live attenuated viruses are known in the art.

In one embodiment, the drug product comprises the same components as the TFF retentate so that it is not necessary to remove compounds used in the purification process after the purification process. Hence, in one embodiment, the drug product comprises the infectious viral particles, non-ionic surfactant, sugar and protein. Preferably, the drug product comprises the infectious viral particles, poloxamer 407, trehalose dihydrate and human serum albumin. More preferably, the drug product comprises the infectious dengue viral particles, poloxamer 407, trehalose dihydrate and human serum albumin. Most preferably, the drug product comprises the infectious dengue viral particles of all four dengue serotypes, poloxamer 407, trehalose dihydrate and human serum albumin.

### EXAMPLES

The following Examples are included to demonstrate certain aspects and embodiments of the invention as described in the claims. It should be appreciated by those of skill in the art, however, that the following description is illustrative only and should not be taken in any way as a restriction of the invention.

### Example 1: Preparation of the Dengue Virus Strains

The methods used to generate the chimeric dengue strains TDV-1, -3 and -4 were standard molecular cloning and DNA engineering methods and are described in Huang et al. (2003) J. Virology 77(21): 11436-11447. The following well-known methods were used to construct and introduce the prM-E genes of dengue serotypes 1, 3 and 4 into the TDV-2 backbone: Reverse-transcriptase PCR (RT-PCR), PCR, restriction enzyme digestion, DNA fragment ligation, bacterial transformations by electroporation, plasmid DNA preparations, in vitro transcription by T7 RNA polymerase, and transfection of Vero cells by electroporation. The different dengue serotypes were grown separately as described in Huang et al. (2013) PLOS Neglected Dis, 7(5):e2243.

### Example 2: Purification of the Dengue Virus Strains

The Vero cell supernatant was collected on days 5, 6, 7, 8, 9 and 10 after infection and new medium was added to the Vero cells on days 5, 6, 7, 8 and 9. Each daily harvest was clarified using a Sartopore 2 filter with 0.45+0.2 μm pore size and 0.1 m² membrane area.

The clarified harvest was mixed with a solution comprising 3% poloxamer 407, 45% trehalose dihydrate and 0.3% human serum albumin (“3×FTA”) wherein one part of 3×FTA was mixed with 14 parts of the clarified harvest so that the final concentration of FTA was 0.2×(0.2% poloxamer 407, 3% trehalose dihydrate and 0.02% human serum albumin). The mixture was applied to Sartobind Q SingleSep nano membrane which has stabilized reinforced cellulose as base material and quaternary ammonium groups as ion exchange groups. The anion exchange chromatography was run in flow-through mode and the flow-through was collected. Afterwards a TFF step using a Hydrosart membrane with a membrane cut-off of 100 kDa was performed with a feed flow rate of 300 LMH and a transmembrane pressure of 0.35 to 0.40 bar. The membrane was flushed two times with a buffer containing 1% poloxamer 407, 27% trehalose and 0.1% HSA and the retentate was filtered through a Sartopore 2 filter with pore size 0.45+0.2 μm. The sample obtained by this method is designated as “0.2×FTA stabilized harvest (DMV008-2)” in Table 2 below.

In further experiments, the clarified harvest was directly subjected to anion exchange chromatography without mixing the clarified harvest with FTA. The clarified harvest was applied to Sartobind Q SingleSep nano membrane which has stabilized reinforced cellulose as base material and quaternary ammonium groups as ion exchange groups. The anion exchange chromatography was run in flow-through mode and the flow-through was collected and the host cell DNA content was determined. This sample is designated as “Clarified harvest (DMV005-3)” in Table 2 below.

The host cell DNA content was determined by quantitative PCR using primers which specifically bind to Vero cell DNA, but not to the viral nucleic acids and the Picogreen® dye. The virus titer was determined by an immunofocus assay.

TABLE 2

| | | HC DNA | Log₁₀ |
|---|---|---|---|
| Feed | | Concentration | total Titer |
| Material | Samples | (ng/mL) ^{a} | (PFU) |
| Clarified | Feed | 779.4 | 8.59 |
| harvest | Flow-through | ≤50 | 7.25 |
| (DMV005-3) | Reduction | ≥729 | 1.33 |
| 0.2XFTA | Feed | 1174.79 | 10.46 |
| stabilized | Flow-through | ≤50 | 10.37 |
| harvest | Reduction | ≥1124 | 0.09 |
| (DMV008-2) | | | |

Table 2 shows that anion exchange chromatography greatly reduces the host cell DNA concentration in the sample, as the flow-through from the anion exchange chromatography membrane comprises ≤50 ng/ml host cell DNA which is below the detection limit of the assay used. However, there were great differences in the virus titer after anion exchange chromatography depending on the feed material used. A strong loss of virus titer, i.e. a reduction by 1.33 log₁₀ PFU, was observed when the clarified harvest was subjected to anion exchange chromatography without mixing the clarified harvest with FTA to a final concentration of 0.2×FTA. In contrast, when the anion exchange chromatography was performed after the clarified harvest had been mixed with FTA to a final concentration of 0.2×FTA, there was only a minimal loss of virus titer.

### LIST OF ITEMS OF THE INVENTION

1. Method for removing host cell DNA from a sample comprising infectious viral particles and host cell DNA, comprising the steps of:
   (a) mixing said sample comprising infectious viral particles and host cell DNA with a composition comprising one or more of a non-ionic surfactant, a sugar and a protein, thereby providing a mixture; and
   (b) subjecting the mixture of step (a) to anion exchange chromatography.
2. Method according to item 1, wherein the non-ionic surfactant is a nonionic triblock copolymer.
3. Method according to item 1 or 2, wherein the non-ionic surfactant is an ethylene oxide propylene oxide (EO-PO) block copolymer.
4. Method according to any one of the preceding items, wherein the non-ionic surfactant is poloxamer 407 or poloxamer 403.
5. Method according to any one of the preceding items, wherein the concentration of the non-ionic surfactant in the mixture of step (a) is 0.05% to 0.5% (w/v).
6. Method according to any one of the preceding items, wherein the sugar is selected from trehalose, glucose, sucrose, fructose and maltose, preferably is trehalose.
7. Method according to any one of the preceding items, wherein the concentration of the sugar in the mixture of step (a) is 1% to 7.5% (w/v).
8. Method according to any one of the preceding items, wherein the protein is an albumin, preferably is human serum albumin.
9. Method according to any one of the preceding items, wherein the concentration of the protein in the mixture of step (a) is 0.005% to 0.05% (w/v).
10. Method according to any one of the preceding items, wherein the composition comprises poloxamer 407, trehalose dihydrate and human serum albumin.
11. Method according to item 10, wherein the mixture of step (a) comprises 0.2% (w/v) poloxamer 407, 3% trehalose dihydrate and 0.02% human serum albumin.
12. Method according to any one of the preceding items, wherein the anion exchange chromatography is operated in flow-through mode.
13. Method according to any one of the preceding items, wherein the anion exchange chromatography uses an anion exchange chromatography membrane.
14. Method according to item 13, wherein the anion exchange chromatography membrane comprises quaternary ammonium groups.
15. Method according to any one of the preceding items, wherein the infectious viral particles are enveloped infectious viral particles.
16. Method according to any one of the preceding items, wherein the infectious viral particles are infectious flavivirus viral particles.
17. Method according to any one of the preceding items, wherein the infectious viral particles are infectious dengue virus viral particles.
18. Method according to item 17, wherein the infectious dengue virus viral particles are infectious live attenuated dengue virus viral particles or infectious chimeric dengue virus viral particles.
19. Method according to any one of the preceding items, wherein less than 5% of the viral titer are lost during the anion exchange chromatography.
20. Method according to any one of the preceding items, wherein the anion exchange chromatography results in an at least tenfold reduction of the host cell DNA content.
21. Method according to any one of the preceding items, wherein the method does not include a step of treating the sample comprising infectious viral particles and host cell DNA with an endonuclease.
22. Method for removing host cell DNA from a sample comprising infectious dengue virus viral particles and host cell DNA, comprising the steps of:
   (a) mixing said sample comprising infectious dengue virus viral particles and host cell DNA with a composition comprising one or more of a nonionic triblock copolymer, a non-reducing sugar and albumin, thereby providing a mixture; and
   (b) subjecting the mixture of step (a) to anion exchange chromatography.
23. Method for removing host cell DNA from a sample comprising infectious dengue viral particles and host cell DNA, comprising the steps of:
   (a) mixing said sample comprising infectious dengue virus viral particles and host cell DNA with a composition comprising a nonionic triblock copolymer, a non-reducing sugar and albumin, thereby providing a mixture; and
   (b) subjecting the mixture of step (a) to anion exchange chromatography.
24. Method according to item 22 or 23, wherein the nonionic triblock copolymer is an ethylene oxide propylene oxide (EO-PO) block copolymer.
25. Method according to any one of items 22 to 24, wherein the nonionic triblock copolymer is poloxamer 407 or poloxamer 403.
26. Method according to any one of items 22 to 25, wherein the concentration of the nonionic triblock copolymer in the mixture of step (a) is 0.05% to 0.5% (w/v).
27. Method according to any one of items 22 to 26, wherein the non-reducing sugar is trehalose.
28. Method according to any one of items 22 to 27, wherein the concentration of the non-reducing sugar in the mixture of step (a) is 1% to 7.5% (w/v).
29. Method according to any one of items 22 to 28, wherein the albumin is human serum albumin.
30. Method according to any one of any one of items 22 to 29, wherein the concentration of the albumin in the mixture of step (a) is 0.005% to 0.05% (w/v).
31. Method according to any one of items 22 to 30, wherein the composition comprises poloxamer 407, trehalose dihydrate and human serum albumin.
32. Method according to item 31, wherein the mixture of step (a) comprises 0.2% (w/v) poloxamer 407, 3% trehalose dihydrate and 0.02% human serum albumin.
33. Method according to any one of items 22 to 32, wherein the anion exchange chromatography is operated in flow-through mode.
34. Method according to any one of items 22 to 33, wherein the anion exchange chromatography uses an anion exchange chromatography membrane.
35. Method according to item 34, wherein the anion exchange chromatography membrane comprises quaternary ammonium groups.
36. Method according to any one of items 22 to 35, wherein the infectious dengue virus viral particles are infectious live attenuated dengue virus viral particles or infectious chimeric dengue virus viral particles.
37. Method according to any one of items 22 to 36, wherein less than 5% of the viral titer are lost during the anion exchange chromatography.
38. Method according to any one of items 22 to 37, wherein the anion exchange chromatography results in an at least tenfold reduction of the host cell DNA content.
39. Method according to any one of items 22 to 38, wherein the method does not include a step of treating the sample comprising infectious viral particles and host cell DNA with an endonuclease.
40. Method for removing host cell DNA from a sample comprising infectious dengue virus viral particles and host cell DNA, comprising the steps of:
   (a) mixing said sample comprising infectious dengue virus viral particles and host cell DNA with a composition comprising 0.2% (w/v) poloxamer 407, 3% trehalose dihydrate and 0.02% human serum albumin, thereby providing a mixture; and
   (b) subjecting the mixture of step (a) to anion exchange chromatography.
41. Method according to item 40, wherein the anion exchange chromatography is operated in flow-through mode.
42. Method according to item 40 or 41, wherein the anion exchange chromatography uses an anion exchange chromatography membrane.
43. Method according to item 42, wherein the anion exchange chromatography membrane comprises quaternary ammonium groups.
44. Method according to any one of items 40 to 43, wherein the infectious dengue virus viral particles are infectious live attenuated dengue virus viral particles or infectious chimeric dengue virus viral particles.
45. Method according to any one of items 40 to 44, wherein less than 5% of the viral titer are lost during the anion exchange chromatography.
46. Method according to any one of items 40 to 45, wherein the anion exchange chromatography results in an at least tenfold reduction of the host cell DNA content.
47. Method according to any one of items 40 to 46, wherein the method does not include a step of treating the sample comprising infectious dengue virus viral particles and host cell DNA with an endonuclease.
48. Method for removing host cell DNA from a sample comprising infectious dengue virus viral particles and host cell DNA, comprising the steps of:
   (a) mixing said sample comprising infectious dengue virus viral particles and host cell DNA with a composition comprising a nonionic triblock copolymer, a non-reducing sugar and albumin, thereby providing a mixture; and
   (b) subjecting the mixture of step (a) to anion exchange chromatography using an anion exchange membrane comprising quaternary ammonium groups in flow-through mode.
49. Method according to item 48, wherein the nonionic triblock copolymer is an ethylene oxide propylene oxide (EO-PO) block copolymer.
50. Method according to item 48 or 49, wherein the nonionic triblock copolymer is poloxamer 407 or poloxamer 403.
51. Method according to any one of items 48 to 50, wherein the concentration of the nonionic triblock copolymer in the mixture of step (a) is 0.05% to 0.5% (w/v).
52. Method according to any one of items 48 to 51, wherein the non-reducing sugar is trehalose.
53. Method according to any one of items 48 to 52, wherein the concentration of the non-reducing sugar in the mixture of step (a) is 1% to 7.5% (w/v).
54. Method according to any one of items 48 to 53, wherein the albumin is human serum albumin.
55. Method according to any one of any one of items 48 to 54, wherein the concentration of the albumin in the mixture of step (a) is 0.005% to 0.05% (w/v).
56. Method according to any one of items 48 to 55, wherein the composition comprises 35 poloxamer 407, trehalose dihydrate and human serum albumin.
57. Method according to item 56, wherein the mixture of step (a) comprises 0.2% (w/v) poloxamer 407, 3% trehalose dihydrate and 0.02% human serum albumin.
58. Method for removing host cell DNA from a sample comprising infectious dengue virus viral particles and host cell DNA, comprising the steps of:
   (a) mixing said sample comprising infectious dengue virus viral particles and host cell DNA with a composition comprising 0.2% (w/v) poloxamer 407, 3% trehalose dihydrate and 0.02% human serum albumin, thereby providing a mixture; and
   (b) subjecting the mixture of step (a) to anion exchange chromatography using an anion exchange membrane comprising quaternary ammonium groups in flow-through mode.
59. Method for purifying infectious dengue virus viral particles from a host cell culture, comprising the steps of:
   (a) harvesting the host cell culture supernatant containing infectious dengue virus viral particles from said host cell culture;
   (b) mixing the harvested infectious dengue virus viral particles with a composition comprising one or more of a nonionic triblock copolymer, a non-reducing sugar and albumin, thereby providing a mixture;
   (c) subjecting the mixture of step (b) to one or more anion exchange chromatography steps and collecting the flow-through;
   (d) subjecting the flow-through from step (c) to tangential flow filtration and collecting the retentate; and
   (e) recovering the purified infectious dengue virus viral particles from the retentate of step (d).
60. Method for purifying infectious dengue virus viral particles from a host cell culture, comprising the steps of:
   (a) harvesting the host cell culture supernatant containing infectious dengue virus viral particles from said host cell culture;
   (b) mixing the harvested infectious dengue virus viral particles with a composition comprising a nonionic triblock copolymer, a non-reducing sugar and albumin, thereby providing a mixture;
   (c) subjecting the mixture of step (b) to one or more anion exchange chromatography steps and collecting the flow-through;
   (d) subjecting the flow-through from step (c) to tangential flow filtration and collecting the retentate; and
   (e) recovering the purified infectious dengue virus viral particles from the retentate of step (d).
61. Method according to item 59 or 60, wherein the non-ionic triblock copolymer is an ethylene oxide propylene oxide (EO-PO) block copolymer.
62. Method according to any one of items 59 to 61, wherein the non-ionic triblock copolymer is poloxamer 407 or poloxamer 403.
63. Method according to any one of items 59 to 62, wherein the concentration of the non-ionic triblock copolymer in the mixture of step (a) is 0.05% to 0.5% (w/v).
64. Method according to any one of items 59 to 63, wherein the non-reducing sugar is trehalose.
65. Method according to any one items 59 to 64, wherein the concentration of the non-reducing sugar in the mixture of step (a) is 1% to 7.5% (w/v).
66. Method according to any one of items 59 to 65, wherein the albumin is human serum albumin.
67. Method according to any one of items 59 to 66, wherein the concentration of the albumin in the mixture of step (a) is 0.005% to 0.05% (w/v).
68. Method according to any one of items 59 to 67, wherein the composition comprises poloxamer 407, trehalose and human serum albumin.
69. Method according to item 68, wherein the composition comprises 0.2% (w/v) poloxamer 407, 3% trehalose dihydrate and 0.02% human serum albumin.
70. Method according to any one of items 59 to 69 wherein the anion exchange chromatography uses an anion exchange chromatography membrane.
71. Method according to item 70, wherein the anion exchange chromatography membrane comprises quaternary ammonium groups.
72. Method according to any one of items 59 to 71, wherein the infectious dengue virus viral particles are infectious live attenuated dengue virus viral particles or infectious chimeric dengue virus viral particles.
73. Method according to any one of items 59 to 72, wherein the method does not include a step of treating the sample comprising infectious dengue virus viral particles and host cell DNA with an endonuclease.
74. Method according to any one of items 59 to 73, wherein the host cell culture is a Vero cell culture.
75. Method according to any one of items 59 to 74, wherein after harvesting the host cell culture supernatant comprising the infectious dengue virus viral particles is subjected to depth filtration.
76. Method according to item 75, wherein the depth filtration is through a 0.45 μm and a 0.2 μm filter.
77. Method according to any one of items 59 to 76, wherein tangential flow filtration comprises filtration through one or more TFF membrane cassettes.
78. Method according to item 77, wherein the one or more TFF membrane cassettes comprise a cellulose media.
79. Method for purifying infectious dengue virus viral particles from a host cell culture, comprising the steps of:
   (a) harvesting the host cell culture supernatant containing infectious dengue virus viral particles from said host cell culture;
   (b) mixing the harvested infectious dengue virus viral particles with a composition comprising 0.2% (w/v) poloxamer 407, 3% trehalose dihydrate and 0.02% human serum albumin, thereby providing a mixture;
   (c) subjecting the mixture of step (b) to one or more anion exchange chromatography steps and collecting the flow-through;
   (d) subjecting the flow-through from step (c) to tangential flow filtration and collecting the retentate; and
   (e) recovering the purified infectious dengue virus viral particles from the retentate of step (d).
80. Method according to item 79 wherein the anion exchange chromatography uses an anion exchange chromatography membrane.
81. Method according to item 80, wherein the anion exchange chromatography membrane comprises quaternary ammonium groups.
82. Method according to any one of items 79 to 81, wherein the infectious dengue virus viral particles are infectious live attenuated dengue virus viral particles or infectious chimeric dengue virus viral particles.
83. Method according to any one of items 79 to 82, wherein the method does not include a step of treating the sample comprising infectious dengue virus viral particles and host cell DNA with an endonuclease.
84. Method according to any one of items 79 to 83, wherein the host cell culture is a Vero cell culture.
85. Method according to any one of items 79 to 84, wherein after harvesting the host cell culture supernatant comprising the infectious dengue virus viral particles is subjected to depth filtration.
86. Method according to item 85, wherein the depth filtration is through a 0.45 μm and a 0.2 μm filter.
87. Method according to any one of items 79 to 86, wherein tangential flow filtration comprises filtration through one or more TFF membrane cassettes.
88. Method according to item 87, wherein the one or more TFF membrane cassettes comprise a cellulose media.
89. Method for purifying infectious dengue virus viral particles from a host cell culture, comprising the steps of:
   (a) harvesting the host cell culture supernatant containing infectious dengue virus viral particles from said host cell culture;
   (b) mixing the harvested infectious dengue virus viral particles with a composition comprising a nonionic triblock copolymer, a non-reducing sugar and albumin, thereby providing a mixture;
   (c) subjecting the mixture of step (b) to one or more anion exchange chromatography steps using an anion exchange membrane comprising quaternary ammonium groups in flow-through mode and collecting the flow-through;
   (d) subjecting the flow-through from step (c) to tangential flow filtration and collecting the retentate; and
   (e) recovering the purified infectious dengue virus viral particles from the retentate of step (d).
90. Method according to item 89, wherein the non-ionic triblock copolymer is an ethylene oxide propylene oxide (EO-PO) block copolymer.
91. Method according to item 89 or 90, wherein the non-ionic triblock copolymer is poloxamer 407 or poloxamer 403.
92. Method according to any one of items 89 to 91, wherein the concentration of the non-ionic triblock copolymer in the mixture of step (a) is 0.05% to 0.5% (w/v).
93. Method according to any one of items 89 to 92, wherein the non-reducing sugar is trehalose.
94. Method according to any one items 89 to 93, wherein the concentration of the non-reducing sugar in the mixture of step (a) is 1% to 7.5% (w/v).
95. Method according to any one of items 89 to 94, wherein the albumin is human serum albumin.
96. Method according to any one of items 89 to 95, wherein the concentration of the albumin in the mixture of step (a) is 0.005% to 0.05% (w/v).
97. Method according to any one of items 89 to 96, wherein the composition comprises poloxamer 407, trehalose and human serum albumin.
98. Method according to item 97, wherein the composition comprises 0.2% (w/v) poloxamer 407, 3% trehalose dihydrate and 0.02% human serum albumin.
99. Method according to any one of items 89 to 98, wherein the infectious dengue virus viral particles are infectious live attenuated dengue virus viral particles or infectious chimeric dengue virus viral particles.
100. Method according to any one of items 89 to 99, wherein the method does not include a step of treating the sample comprising infectious dengue virus viral particles and host cell DNA with an endonuclease.
101. Method according to any one of items 89 to 100, wherein the host cell culture is a Vero cell culture.
102. Method according to any one of items 89 to 101, wherein after harvesting the host cell culture supernatant comprising the infectious dengue virus viral particles is subjected to depth filtration.
103. Method according to item 102, wherein the depth filtration is through a 0.45 μm and a 0.2 μm filter.
104. Method according to any one of items 89 to 103, wherein tangential flow filtration comprises filtration through one or more TFF membrane cassettes.
105. Method according to item 104, wherein the one or more TFF membrane cassettes comprise a cellulose media.
106. Method for purifying infectious dengue virus viral particles from a Vero host cell culture, comprising the steps of:
   (a) harvesting the Vero host cell culture supernatant containing infectious dengue virus viral particles from said Vero host cell culture;
   (b) mixing the harvested infectious dengue virus viral particles with a composition comprising a nonionic triblock copolymer, a non-reducing sugar and albumin, thereby providing a mixture;
   (c) subjecting the mixture of step (b) to one or more anion exchange chromatography steps using an anion exchange membrane comprising quaternary ammonium groups in flow-through mode and collecting the flow-through;
   (d) subjecting the flow-through from step (c) to tangential flow filtration through one or more TFF membrane cassettes comprising a cellulose media and collecting the retentate; and
   (e) recovering the purified infectious dengue virus viral particles from the retentate of step (d).
107. Method according to item 106, wherein the non-ionic triblock copolymer is an ethylene oxide propylene oxide (EO-PO) block copolymer.
108. Method according to item 106 or 107, wherein the non-ionic triblock copolymer is poloxamer 407 or poloxamer 403.
109. Method according to any one of items 106 to 108, wherein the concentration of the non-ionic triblock copolymer in the mixture of step (a) is 0.05% to 0.5% (w/v).
110. Method according to any one of items 106 to 109, wherein the non-reducing sugar is trehalose.
111. Method according to any one items 106 to 110, wherein the concentration of the non-reducing sugar in the mixture of step (a) is 1% to 7.5% (w/v).
112. Method according to any one of items 106 to 111, wherein the albumin is human serum albumin.
113. Method according to any one of items 106 to 112, wherein the concentration of the albumin in the mixture of step (a) is 0.005% to 0.05% (w/v).
114. Method according to any one of items 106 to 113, wherein the composition comprises poloxamer 407, trehalose and human serum albumin.
115. Method according to item 114, wherein the composition comprises 0.2% (w/v) poloxamer 407, 3% trehalose dihydrate and 0.02% human serum albumin.
116. Method according to any one of items 106 to 115, wherein the infectious dengue virus viral particles are infectious live attenuated dengue virus viral particles or infectious chimeric dengue virus viral particles.
117. Method according to any one of items 106 to 116, wherein the method does not include a step of treating the sample comprising infectious dengue virus viral particles and host cell DNA with an endonuclease.
118. Method according to any one of items 106 to 117, wherein after harvesting the host cell culture supernatant comprising the infectious dengue virus viral particles is subjected to depth filtration.
119. Method according to item 118, wherein the depth filtration is through a 0.45 μm and a 0.2 μm filter.
120. Method for purifying infectious dengue virus viral particles from a Vero host cell culture, comprising the steps of:
   (a) harvesting the Vero host cell culture supernatant containing infectious dengue virus viral particles from said Vero host cell culture;
   (b) mixing the harvested infectious dengue virus viral particles with a composition comprising 0.2% (w/v) poloxamer 407, 3% trehalose dihydrate and 0.02% human serum albumin, thereby providing a mixture;
   (c) subjecting the mixture of step (b) to one or more anion exchange chromatography steps using an anion exchange membrane comprising quaternary ammonium groups in flow-through mode and collecting the flow-through;
   (d) subjecting the flow-through from step (c) to tangential flow filtration through one or more TFF membrane cassettes comprising a cellulose media and collecting the retentate; and
   (e) recovering the purified infectious dengue virus viral particles from the retentate of step (d).
121. Method according to item 120, wherein the infectious dengue virus viral particles are infectious live attenuated dengue virus viral particles or infectious chimeric dengue virus viral particles.
122. Method according to item 120 or 121, wherein the method does not include a step of treating the sample comprising infectious dengue virus viral particles and host cell DNA with an endonuclease.
123. Method according to any one of items 120 to 122, wherein after harvesting the host cell culture supernatant comprising the infectious dengue virus viral particles is subjected to depth filtration.
124. Method according to item 123, wherein the depth filtration is through a 0.45 μm and a 0.2 μm filter.

1. Method for removing host cell DNA from a sample comprising infectious viral particles and host cell DNA, comprising the steps of:
(a) mixing said sample comprising infectious viral particles and host cell DNA with a composition comprising one or more of a non-ionic surfactant, a sugar and a protein, thereby providing a mixture; and
(b) subjecting the mixture of step (a) to anion exchange chromatography.

2. Method according to claim 1, wherein the non-ionic surfactant is poloxamer 407 or poloxamer 403.

3. Method according to claim 1, wherein the sugar is selected from trehalose, glucose, sucrose, fructose and maltose, preferably is trehalose.

4. Method according to claim 1, wherein the protein is an albumin, preferably is human serum albumin.

5. Method according to claim 1, wherein the composition comprises poloxamer 407, trehalose dihydrate and human serum albumin.

6. Method according to claim 1, wherein the anion exchange chromatography is operated in flow-through mode.

7. Method according to claim 1, wherein the anion exchange chromatography uses an anion exchange chromatography membrane.

8. Method according to claim 1, wherein the infectious viral particles are infectious dengue virus viral particles.

9. Method for purifying infectious viral particles from a host cell culture, comprising the steps of:
(a) harvesting the host cell culture supernatant containing infectious viral particles from said host cell culture;
(b) mixing the harvested infectious viral particles with a composition comprising one or more of a non-ionic surfactant, a sugar and a protein, thereby providing a mixture;
(c) subjecting the mixture of step (b) to one or more anion exchange chromatography steps and collecting the flow-through;
(d) subjecting the flow-through from step (c) to tangential flow filtration and collecting the retentate; and
(e) recovering the purified infectious viral particles from the retentate of step (d).

10. Method according to claim 9, wherein the non-ionic surfactant is poloxamer 407 or poloxamer 403.

11. Method according to claim 9, wherein the sugar is selected from trehalose, glucose, sucrose, fructose and maltose, preferably is trehalose.

12. Method according to claim 9, wherein the protein is an albumin, preferably is human serum albumin.

13. Method according to claim 9, wherein the composition comprises poloxamer 407, trehalose and human serum albumin.

14. Method according to claim 9, wherein the anion exchange chromatography uses an anion exchange chromatography membrane.

15. Method according to claim 9, wherein the infectious viral particles are infectious dengue virus viral particles.

16. Method according to claim 9, wherein the host cell culture is a Vero cell culture.

17. Method according to claim 9, wherein after harvesting the host cell culture supernatant comprising the infectious viral particles is subjected to depth filtration.

18. Method according to claim 9, wherein tangential flow filtration comprises filtration through one or more TFF membrane cassettes.
